(19) United States
(12) Patent Application Publication
(10) Pub. No.: US 2011/0262561 A1
(43) Pub. Date: Oct. 27, 2011

(54) Protoilludance Norsesquiterpenoid Esters and Uses Thereof

(75) Inventors: Chien-Chih Chen, Taipei City (TW); Jing-Jy Cheng, New Taipei City (TW); Chien-Chang Shen, Taipei City (TW)

(73) Assignee: NATIONAL RESEARCH INSTITUTE OF CHINESE MEDICINE, Taipei (TW)

(21) Appl. No.: 13/093,080

(22) Filed: Apr. 25, 2011

Related U.S. Application Data
(60) Provisional application No. 61/327,893, filed on Apr. 26, 2010.

(51) Int. Cl. A61K 33/24; A61K 31/235; A61K 31/513; A61P 35/02; A61K 31/704; C07C 67/58; A61P 35/00; C07C 69/94; A61K 31/437

(52) U.S. Cl. 424/649; 560/63; 514/510; 514/274; 514/283; 514/34

(57) ABSTRACT

Disclosed herein are novel protoilludane norsesquiterpenoid ester compounds isolated from mycelium of Armillaria mellea that are useful for treating tumors or proliferative diseases such as breast cancers, lung cancers, colon cancers or leukemia.

### RELATED APPLICATIONS

This application claims priority to U.S. Provisional Application Ser. No. 61/327,893, filed Apr. 26, 2010, which is herein incorporated by reference.

### TECHNICAL FIELD

This disclosure in general relates to pharmacology field, and more particularly, relates to protoilludance norsesquilterpenoid esters isolated form mycelium of Armillaria mellea and their uses as therapeutic agents.

### BACKGROUND ART

### Description of Related Art

Armillaria mellea (Tricholomataceae) is a medicinal fungus symbiotic with Chinese medicinal herb “Tianma” (Gastrodia elata Blume). The fruiting bodies of Armillaria mellea have been used in traditional Chinese medicine for the treatment of hypertension, headache, insomnia, dizziness, and vertigo.

N⁶-(5-hydroxy-2-pyridylmethylamino)-9-β-D-ribofuranosylpurine with cerebral-protecting activity has been isolated from the mycelium of A. mellea. (Lee et al., (1998) Chinese Journal of Medicinal Chemistry 8(2): 116; Junhua et al., (1990) Fitoterapia 61:207-214; and Watanabe et al., (1990) Planta Med 56:48-52). Previous chemical studies of the mycelium of A. mellea have also identified a number of sesquiterpenoid aromatic esters having a protoilludane skeleton (Watanabe et al., supra; Yang et al., (1984) Planta Med 50:288-290; Yang et al., (1989) Planta Med 55:479-481; Yang et al., (1989) Planta Med 55:564-565; Obuchi et al., (1989) Planta Med 56:198-201; and Yang et al., (1991) Planta Med 57:478-480). Some of these sesquiterpenoids have been demonstrated to exhibit antimicrobial activities against gram-positive bacteria and yeast (Obuchi et al., supra).

Since mycelium of A. mellea may be artificially cultured in large scale using liquid broth, therefore, it may serve as an abundance source for producing the therapeutic sesquiterpenoids as described above. This invention addresses such need by providing a method of isolating therapeutic sesquiterpenoids from mycelium of A. mellea, and total of 17 active compounds have been identified, among them, 7 novel compounds are found, and all these novel compounds are potential lead compounds for use as anti-angiogenic agents.

### SUMMARY

As embodied and broadly described herein, disclosure herein features protoilludane norsesquiterpenoid esters isolated from mycelium of Armillaria mellea and their uses as anti-angiogenic agents. Total of 17 active compounds are isolated from mycelium of Armillaria mellea by repetitive liquid chromatography, among them, 7 compounds (i.e., mellendonal B, melleolide N, melleolide Q, melleolide P, melleolide R, melleolide S and melleolide T) are novel, and the rest are known compounds. All 17 compounds are subsequently confirmed with anti-angiogenesis activity and are therefore potential lead compounds for use as anti-angiogenic agents.

Accordingly, it is the first aspect of this disclosure to provide a method of isolating a protoilludane norsesquiterpenoid ester compound. The method includes steps of: extracting the mycelia of Armillaria mellea with an alcohol solution; partitioning the alcoholic extract with a mixture of ethyl acetate and water to produce an ethyl acetate layer and an aqueous layer; separating the protoilludane norsesquiterpenoid ester compound from the ethyl acetate layer by liquid chromatography with a mixture of n-hexane and ethyl acetate in a ratio from about 20:1 to about 0:1 (v/v).

In one embodiment, the alcohol solution is ethanol.

The protoilludane norsesquiterpenoid ester compound isolated by the method of this invention has a formula of,

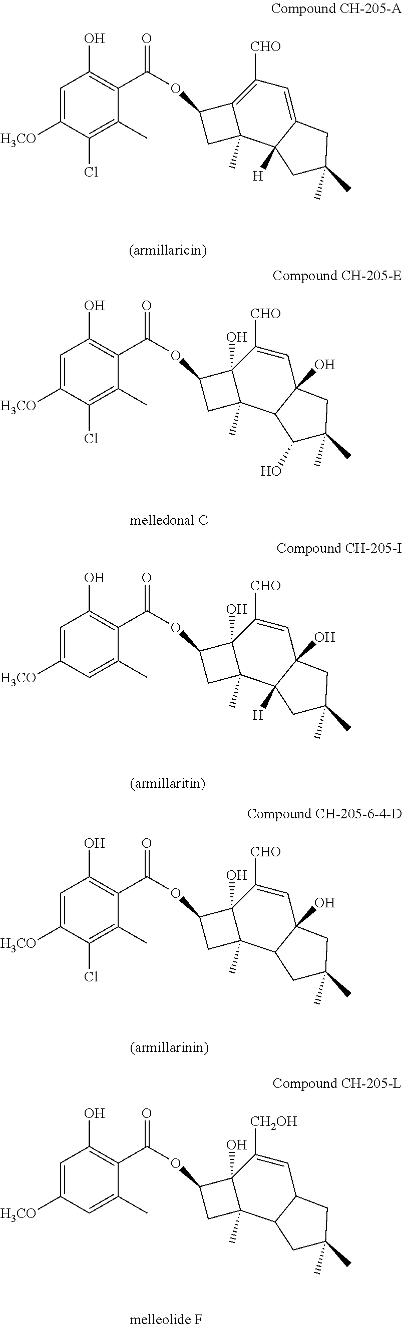

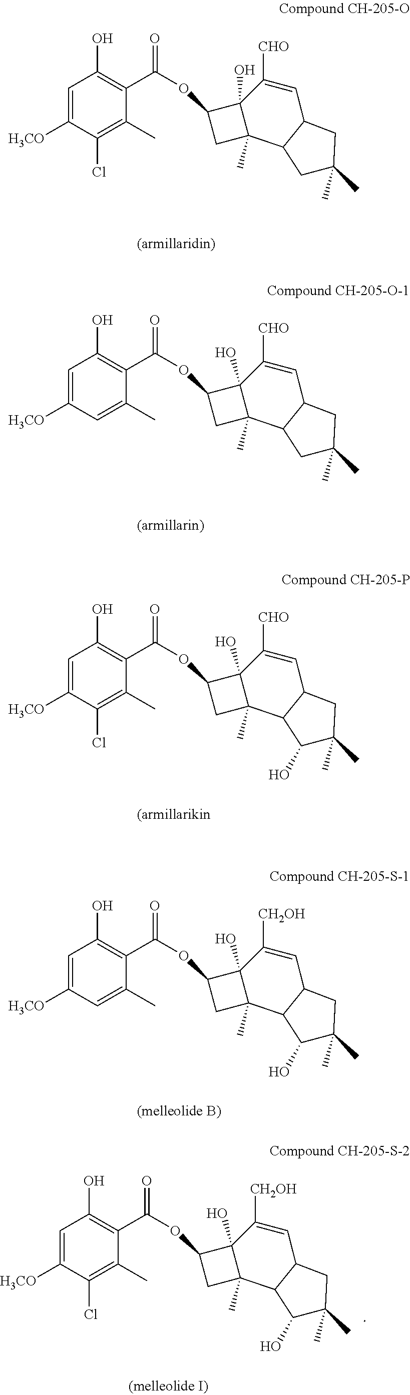

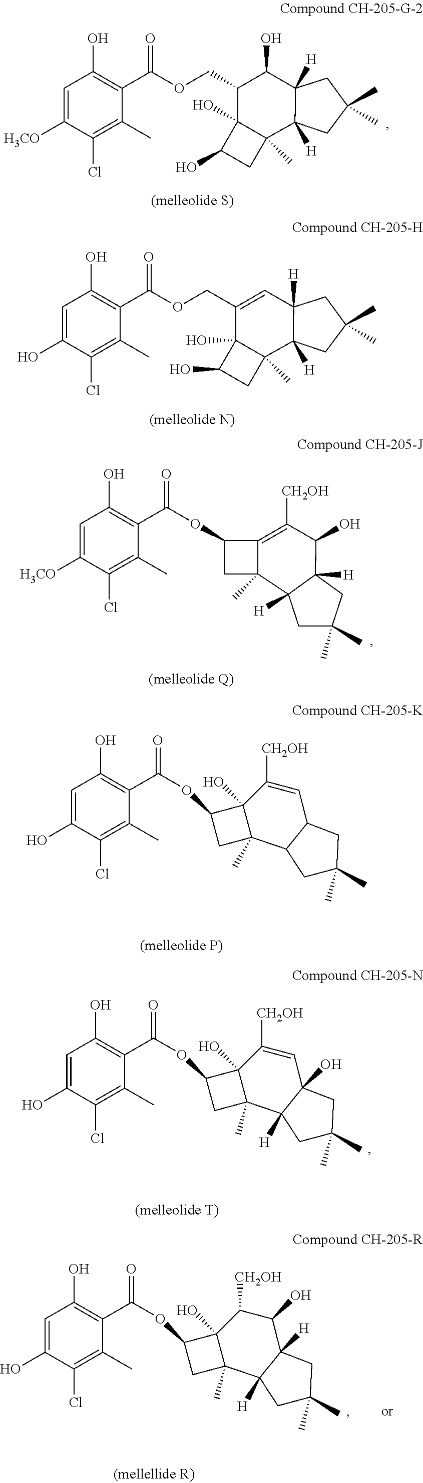

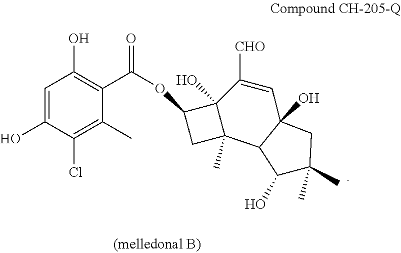

Among the isolated protoilludane norsesquiterpenoid ester compounds, mellendonal B, melleolide N, melleolide Q, melleolide P, melleolide R, melleolide S and melleolide T are novel. Accordingly, the second aspect of this disclosure is related to a pharmaceutical pharmaceutical composition for treating a proliferative disease, such as a tumor. The pharmaceutical composition includes a therapeutically effective amount of the protoilludane norsesquiterpenoid ester compound isolated by the method described above; and a pharmaceutically acceptable excipient, wherein the protoilludane norsesquiterpenoid ester compound possesses anti-angiogenesis activity and is capable of reducing the size of the tumor. In some embodiments, the composition further includes a chemotherapeutic agent selected from the group consisting of 5-fluorouracil, vinblastin, doxorubicin and cisplatin.

It is therefore the third aspect of this disclosure to provide a method of treating a proliferative disease such as a tumor. The method includes a step of administering to a subject in need thereof an effective amount of the protoilludane norsesquiterpenoid ester compound isolated by the method described above, wherein the protoilludane norsesquiterpenoid ester compound possesses anti-angiogeneic activity and is capable of reducing the size of the tumor.

In some embodiments, the tumor or the proliferative disease is any of a breast cancer, a lung cancer, a colon cancer or leukemia.

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 illustrates migration activity of CH-205 compounds on endothelial cells using wound healing assay in accordance with one embodiment of this invention;

FIG. 2A and FIG. 2B illustrates the activity of CH-205-O against the H460 human lung cancer tumor xenograft in accordance with one embodiment of this invention;

FIG. 3 illustrates the tumor inhibition rate of CH-205-O against the H460 human lung cancer tumor xenograf in accordance with one embodiment of this invention;

FIG. 4A and FIG. 4B illustrates the activity of CH-205-K against the H460 human lung cancer tumor xenograft in accordance with one embodiment of this invention; and

FIG. 5 illustrates the tumor inhibition rate of CH-205-K against the H460 human lung cancer tumor xenograft in accordance with one embodiment of this invention.

### DISCLOSURE OF INVENTION

The practices of this invention are hereinafter described in detail with respect to a process of isolating protoilludane norsesquiterpenoid ester compounds from Armillaria mellea and their therapeutic uses.

First, an organic solvent extract of Armillaria mellea is prepared by extracting mycelium with a suitable organic solvent in accordance with any method that is well known in the art. The organic solvent may include, but is not limited to, alcohols (e.g., methanol, ethanol, propanol and isopropanol), esters (e.g., acetic acetate), alkanes (e.g., hexanes and cyclohexanes), alkyl halides (e.g., mono- or di-chloromethane, mono- or di-chloroethane). Preferably, the organic solvent is from alcohols. More preferably, the organic solvent is ethanol.

The organic solvent extract of Armillaria mellea is then partitioned with a mixture of ethyl acetate and water, so as to produce an ethyl acetate layer and an aqueous layer. The protoilludane norsesquiterpenoid esters are then isolated and purified by using high-performance liquid chromatography to obtain various fractions, Each fraction is then tested and confirmed the structure of the active compound(s) therein by UV, IR, MS, ¹H-NMR, ¹³C-NMR and 2D-NMR analysis. The protoilludane norsesquiterpenoid esters isolated from each fraction are tested for their in vitro cytotoxicity effects on cancer cells and inhibitory effects on edothelial cell migration, as well as their in vivo inhibitory effects on the growth of xenograft tumors. For HPLC isolation and purification, a mixture of n-hexane and ethyl acetate in a ratio from about 20:1 to about 0:1 (v/v) may be employed to elute the active compounds out of the column. In one example, total of 10 fractions may be generated by eluting the column with the mixture of n-hexane and ethyl acetate in a ratio of about 20:1, 5:1, 3:1, 1:1 or 0:1. In one example, the ratio of the mixture of n-hexane and ethyl acetate is about 20:1. In another example, the ratio of the mixture of n-hexane and ethyl acetate is about 5:1. In still another example, the ratio of the mixture of n-hexane and ethyl acetate is about 3:1. In still another example, the ratio of the mixture of n-hexane and ethyl acetate is about 1:1. In still another example, the ratio of the mixture of n-hexane and ethyl acetate is about 0:1.

According to one preferred process of this disclosure, the isolated protoilludane norsesquiterpenoid ester compound has a formula of,

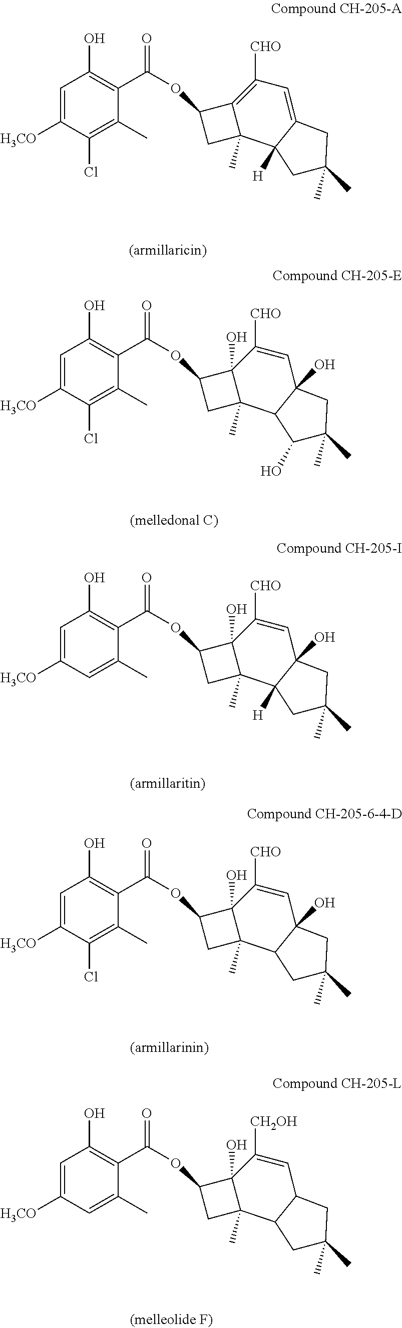

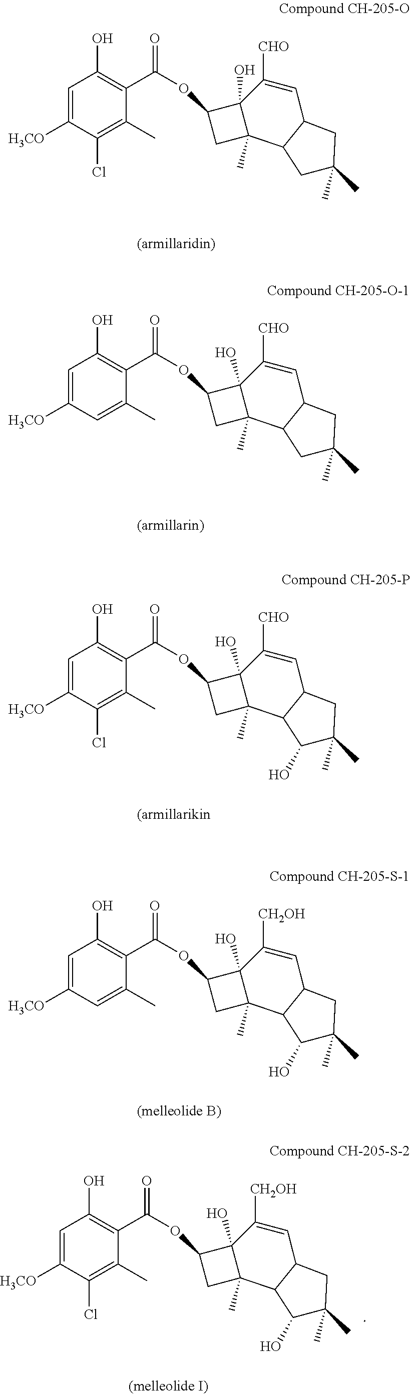

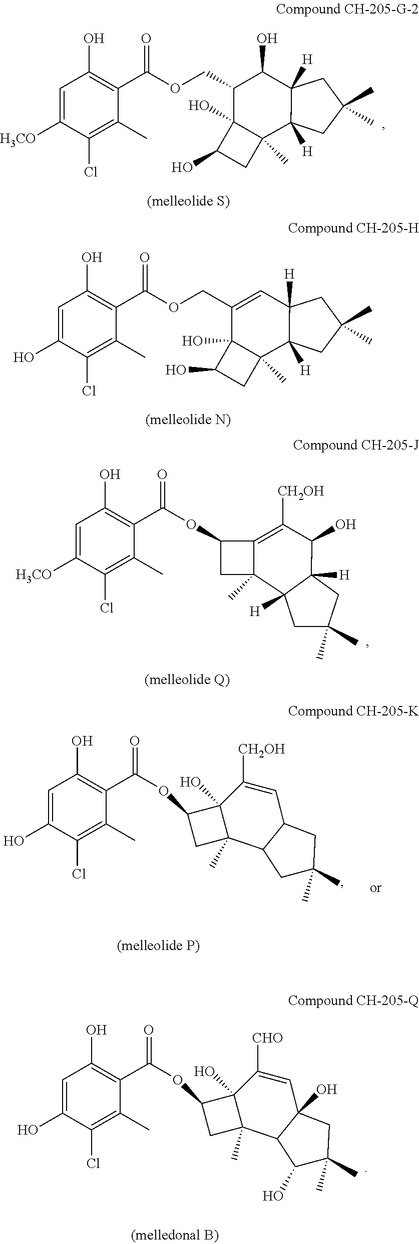

Among these 17 isolated protoilludane norsesquiterpenoid ester compounds, compounds CH-205-G-2 (melleolide S), CH-205-H (melleolide N), CH-205-J (melleolide Q), CH-205-K (melleolide P), CH-205-N (melleolide T), CH-205-Q (melledonal B), and CH-205-R (melleolide R) are new compounds and the rest are known compounds. All compounds isolated in accordance with the as-described method possess in vitro cytotoxity on tumor cells, and anti-angiogenesis activity by inhibiting endothelial cell growth and migration. Further, all the identified compounds may inhibit in vivo growth of a tumor by reducing the size of the xenografted tumor to an extent of at least 30% reduction in tumor size.

Accordingly, it is one objective of this disclosure to provide a pharmaceutical composition for treating a proliferative disease such as a tumor. The composition includes a therapeutically effective amount of a protoilludane norsesquiterpenoid ester compound isolated by the as-described method; and a pharmaceutically acceptable excipient, wherein the protoilludane norsesquiterpenoid ester compound possesses anti-angiogenesis activity and is capable of reducing the size of the tumor for at least about 10%, such as about 10%, 20%, 30%, 40% or 50%, as compared with that of a control.

The isolated protoilludane norsesquiterpenoid ester compounds are confirmed to possess anti-angiogenesis activity.

In one preferred example, the protoilludane norsesquiterpenoid ester compound in the composition is melleolide K (compound CH-205-K).

The pharmaceutical composition may further comprise a chemotherapeutic agent selected from the group consisting of 5-fluorouracil, vinblastin, doxorubicin and cisplatin. In one specific example, the chemotherapeutic agent is cisplatin. In some examples, the proliferative disease is any of a breast cancer, a lung cancer, a colon cancer or leukemia.

The pharmaceutical composition of this disclosure may be administered by oral, parenteral (e.g., intramuscular, intraperitoneal, intravenous or subcutaneous injection or implant), nasal, sublingual, topical or transdermal routes and can be formulated into various dosage forms suitable for each route of administration. A number of suitable dosage forms are described below, but are not meant to include all possible choices. One of skilled person in the art is familiar with the various dosage forms that are suitable for use in each route. It is to be noted that the most suitable route in any given case would depend on the nature or severity of the disease or condition being treated. The active ingredient, such as any of the protoilludane norsesquiterpenoid ester compound isolated and identified as described above, is mixed with at least one pharmaceutically acceptable excipient.

In some embodiments, the pharmaceutical compositions of this disclosure are solid dosage forms for oral administration. Such solid dosage forms may be capsules, sachets, tablets, pills, lozenges, powders or granules. In such forms, the active ingredient such as verapamil is mixed with at least one pharmaceutically acceptable excipient. Any of the described solid dosage forms may optionally contain coatings and shells, such as enteric coatings, and coatings for modifying the release rate of any of the ingredients. Examples of such coatings are well known in the art. In one example, the pharmaceutical compositions of this disclosure are tablets such as quick-release tablets. In still another example, the pharmaceutical compositions of this disclosure are formulated into sustained release forms. In another example, the pharmaceutical compositions of this disclosure are powders that are encapsulated in soft and hard gelatin capsules.

In some embodiments, the pharmaceutical compositions of this disclosure are liquid dosage forms for oral administration. The liquid formulation may further include a buffering agent to maintain a desired pH. The liquid dosage formulations may also be filled into soft gelatin capsules. For example, the liquid may include a solution, suspension, emulsion, microemulsion, precipitate or any desired liquid media carrying any of the protoilludane norsesquiterpenoid ester compound as described above or a pharmaceutically acceptable derivative, salt or solvate thereof, or a combination thereof. The liquid may be designed to improve the solubility of the active compound as described above to form a drug-containing emulsion or disperse phase upon release.

In some embodiments, the pharmaceutical compositions of this disclosure are formulations suitable for parenteral administration, such as administration by injection, which includes, but is not limited to, subcutaneous, bolus injection, intramuscular, intraperitoneal and intravenous injection. The pharmaceutical compositions may be formulated as isotonic suspensions, solutions or emulsions in oily or aqueous vehicles, and may contain formulatoary agents such as suspending, stabilizing or dispersing agents. Alternatively, the compositions may be provided in dry form such as powders, crystallines or freeze-dried solids with sterile pyrogen-free water or isotonic saline before use. They may be presented in sterile ampoules or vials.

In some embodiments, the pharmaceutical compositions of this disclosure are formulations suitable for intranasal administration or administration by inhalation. The compositions are delivered in the form of a solution or suspension from a pump spray container that is squeezed or pumped by the patient or in the form of a dry powder or as an aerosol spray from a pressurized container or a nebulizer, with the use of a suitable propellant such as dichlorodifluoromethane, trichlorofluoromethane, dichlorotetrafluoromethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurized aerosol, the dosage unit may be determined by providing a valve to deliver a metered amount. The pressurized container or nebulizer may contain a solution or suspension of the composition of this disclosure. Capsules or cartridges for use in an inhaler or insufflators may be formulated to contain a powder mix of the active compound of this disclosure.

In some embodiments, the pharmaceutical compositions of this disclosure are dosage formulations that are suitable for topical or transdermal administration. Such formulations include sprays, ointments, pastes, creams, lotions, gels, solutions and patches. Such formulations may optionally include excipients such as animal and vegetable fats, oils, waxes, paraffins, starch, tragacanth, cellulose derivatives, silicons, bentonites, silicic acid, talc, zinc oxide or mixtures thereof. Sprays may also include excipients such as talc, silicic acid, aluminum hydroxide and calcium silicate; additionally, sprays may contain propellants such as chlorofluoro-dydrocarbons and volatile hydrocarbons such as butane and propane. Transdermal patches may be made by dissolving, dispersing or incorporating a pharmaceutical composition of the present disclosure in a suitable medium, such as elastomeric matrix material. Absorption enhancers may also be used to increase the flux of the mixture across the skin. Alternatively, the composition of the present disclosure may be formulated into a lotion or a cream.

It is another objective of this disclosure to provide a method of treating a proliferative disease such as a tumor. The method includes a step of administering to a subject in need thereof an effective amount of a protoilludane norsesquiterpenoid ester compound isolated by the present invention: wherein the protoilludane norsesquiterpenoid ester compound possesses anti-angiogeneic activity and is capable of reducing the size of the tumor for at least 30%.

The tumor or the proliferative disease that may be treated by the afore-mentioned method is any of a breast cancer, a lung cancer, a colon cancer or leukemia. In one example, armillaridin (compound CH-205-O) is used for such treatment. In another example, armillaridin (compound CH-205-O) is used in combination with a chemotherapeutic agent selected from the group consisting of 5-fluorouracil, vinblastin, doxorubicin and cisplatin to treat tumor or proliferative disease.

The detail description of preferred embodiments of the present disclosure is as follows.

### EXAMPLES

### Cell Culture

The endothelial cells (human endothelial-like cells, Eahy926) and 4 cancer cell lines (MCF-7, H460, HT-29, and CEM) were derived from the American Type Culture Collection (ATCC) and were maintained in DMEM or RPMI medium (Life Technologies) supplemented with 2 mM L-glutamine and 10% heat-inactivated fetal bovine serum (FBS) (Life Technologies) under standard culture conditions. The cell viability and cell number were determined by the Trypan Blue dye-exclusion method.

### High-Performance Liquid Chromatography (HPLC)

HPLC was conducted on a HP/Agilent 1100 liquid chromatography equipped with a UV spectrophotometric detector. The crude materials were separated by using a Cosmosil 5C-18 MS-II (5 μm, 10×250 mm) column at room temperature, and four different mobile phase conditions (A-D) were used in the extraction procedure, respectively, and details condition listed in Table 1.

TABLE 1

| | Time | H₂O | Acetonitril |
|---|---|---|---|
| Condition | (min) | (%) | (%) |
| A | 0 | 15 | 85 |
| | 20 | 0 | 100 |
| | 25 | 0 | 100 |
| B | 0 | 50 | 50 |
| | 30 | 0 | 100 |
| C | — | 38 | 62 |
| D | — | 40 | 60 |

### Statistical Analysis

Statistical analysis was performed using the Student's t test. Data are presented as mean±SEM. Statistical significance was defined as P<0.05.

### Example 1

### Extraction and Identification of Protoilludane Norsesquiterpenoid Esters of Armillaria mellea

1.1 Extraction and Isolation of Proteilludane Norsesquiterpenoid Esters of Armillaria mellea

The mycelium of Armillaria mellea (9 kg) (CH-205) was extracted with 95% EtOH for three times. The 95% EtOH soluble portion was concentrated to provide EtOH extract that was partitioned with H₂O and EtOAc. The EtOAc layer was chromatographed on silica gel column and eluted with n-hexane-EtOAc (20:1→0:1) and 10 fractions (Fr-1-Fr-10) were obtained. Fraction Fr-3 (n-hexane/EtOAc=5:1) was crystallized with MeOH to get CH-205-A (114 mg). Fraction Fr-4 (n-hexane/EtOAc=5:1) was purified by semi-preparative reversed-phase HPLC (Condition A) to give three compounds, which were CH-205-O-1 (Rt=17.14 min), CH-205-O (Rt=18.53 min), and CH-205-O-2 (Rt=23.97 min), respectively. Fraction Fr-6 (n-hexane/EtOAc=3:1) was repeatedly chromatographed using silica gel and Sephadex LH-20 columns to obtain compound CH-205-6-4-D (120 mg) and two sub-fractions, Fr-6-1 and Fr-6-2. Sub-fraction Fr-6-1 was further purified by semi-preparative reversed-phase HPLC (Condition B) to give two compounds CH-205-6-4-B-1 (Rt=25.28 min) and CH-205-6-4-B-2 (Rt=30.48 min). Fraction Fr-7 (n-hexane/EtOAc=1:1) was repeatedly chromatographed using silica gel and Sephadex LH-20 columns to obtain five compounds, including CH-205-P (675.5 mg), CH-205-N (42.9 mg), CH-205-K (57.7 mg), CH-205-L, and CH-205-L-1. Fraction 8 (n-hexane/EtOAc=1:1) was repeatedly chromatographed using silica gel and Sephadex LH-20 columns to get three compounds, which were CH-205-U (538 mg), CH-205-V (185.2 mg), and CH-205-W (47.9 mg). Fraction Fr-9 (n-hexane/EtOAc=0:1) was repeatedly chromatographed using silica gel, Sephadex LH-20 and RP-18 columns to afford six compounds, which included CH-205-C (12.6 mg), CH-205-D (16.2 mg), CH-205-J (35.7 mg), CH-205-E (3.504 g), CH-205-H (100.7 mg) and CH-205-I (54.2 mg), and one sub-fraction Fr-9-1. Sub-fraction Fr-9-1 was further purified with RP-18 column [H₂O:MeOH (2:8)] to obtain two compounds CH-205-G-1 (5.5 mg) and CH-205-G-2 (46.3 mg), and one sub-fraction Fr-9-1-1. Sub-fraction Fr-9-1-1 was further purified by semi-preparative reversed-phase HPLC (Condition C) to give two compounds CH-205-G-3-1 (Rt=29.79 min, 8.8 mg) and CH-205-G-3-2 (Rt=31.95 min, 69.6 mg). Fraction Fr-10 (n-hexane/EtOAc=0:1) was repeatedly chromatographed using silica gel, Sephadex LH-20 and RP-18 columns to afford three compounds CH-205-R (595.1 mg), CH-205-Q (118.4 mg) and CH-205-T (308.7 mg), and one sub-fraction Fr-10-1. Sub-fraction Fr-10-1 was further purified by semi-preparative reversed-phase HPLC (Condition D) to give two compounds CH-205-S-1 (Rt=32.34 min, 17.3 mg) and CH-205-S-2 (Rt=34.76 min, 22.7 mg).

### 1.2 Identification of the Isolated Compounds of Example 1.1

Total of 28 compounds were isolated in accordance with the procedures descried in Example 1.1, however, after spectral analysis which includes UV, IR, MS, ¹H-NMR, ¹³C-NMR and 2D-NMR analysis, only 17 compounds were confirmed, among them, 7 compounds (i.e., CH-205-G-2 (melleolide S), CH-205-H (melleolide N), CH-205-J (melleolide Q), CH-205-K (melleolide P),

CH-205-N (melleolide T), CH-205-Q (melledonal B), and CH-205-R (melleolide R)) are new compounds and the rest are known compounds.

Spectral details of the 17 identified compounds are provided as follows: CH-205-H (melleolide N)

(((2R,2aS,4aS,7aS,7bR)-2,2a,4a,5,6,7,7a,7b-octahydro-2,2a-dihydroxy-6,6,7 b-trimethyl-1H-cyclobuta[e]inden-3-yl)methyl
3-chloro-4,6-dihydroxy-2-methylbenzoate)

Colorless solid, mp. 148-149 ° C.; [a]₂₅−25° (c 1.0, MeOH);

UV (MeOH) λmax (log ε) 307 (3.65), 261 (3.91), 213 (4.45) nm;

IR (KBr) νₘₐₓ 3528, 1631, 1592, 1461, 1425, 1310, 1243, 1128, 1081 cm⁻¹;

¹H NMR (acetone-d₆, 500 MHz) δ0.97 (3H, s, CH₃-14 or CH₃-15), 0.98 (3H, s, CH₃-15 or CH₃-14), 1.16 (3H, s, CH₃-8), 1.34-1.49 (4H, m, H-6, H₂-10, and H-12), 1.76 (1H, dd, J=8.5, 10.5 Hz, H-6), 1.84 (1H, dd, J=8.5, 13.0 Hz, H-12), 2.15 (1H, m, H-9), 2.63 (3H, s, CH₃-8′), 2.75 (1H, m, H-13), 4.35 (1H, dd, J=8.5, 15.0 Hz, H-5), 4.95 (1 H, d, J=12.5 Hz, H-1), 5.08 (1 H, d, J=12.8 Hz, H-1), 5.88 (1H, br s, H-3), 6.44 (1H, s, H-4′), 10.76 (1H, s, OH-3′);

¹³C NMR (acetone-d₆, 500 MHz) δ19.7 (CH₃-8′), 22.3 (CH₃-8), 32.1 (CH₃-14 or CH₃-15), 32.3 (CH₃-15 or CH₃-14), 36.4 (C-6), 38.0 (C-7), 38.5 (C-11), 40.2 (C-13), 42.3 (C-10), 45.4 (C-9), 48.3 (C-12), 67.8 (C-1), 76.9 (C-5), 78.1 (C-4), 102.7 (C-4′), 109.1 (C-2′), 114.7 (C-6′), 132.0 (C-2), 136.1 (C-3), 140.4 (C-7′), 158.2 (C-3′), 161.8 (C-5′), 170.5 (C-1′);

ESIMS m/z (%): 459 [M+Na]⁺; and

HRFAB m/z 437.1732 (calcd 437.1731 for C₂₃H₃₀O₆Cl).

CH-205-J (melleolide Q)
(2R,4S,4aR,7aS,7bR)-2,4,4a,5,6,7,7a,7b-octahydro-4-hydroxy-3-(hydroxymethyl)-6,6,7b-trimethyl-1H-cyclobuta[e]inden-2-yl)
3-chloro-6-hydroxy-4-methoxy-2-methylbenzoate

Colorless solid, mp. ; [a]₂₅−112° (c 1.0, MeOH);

UV (MeOH) λmax (log ε) 305 (3.52), 267 (4.07), 213 (4.42) nm;

IR (KBr) νₘₐₓ 3528, 1647, 1603, 1556, 1461, 1401, 1267, 1215, 1108 cm⁻¹;

¹H NMR (CDCl₃, 500 MHz) δ0.94 (3H, s, CH₃-15), 0.99 (3H, s, CH₃-8), 1.03 (3H, s, CH₃-14), 1.14 (1H, t, J=9.5 Hz, H-12), 1.1.29 (1H, m, H-10), 1.41 (1H, dd, J=7.5, 12.5 Hz, H-12), 1.79 (1H, dd, J=7.0, 11.5 Hz, H-12), 1.93 (1H, dd, J=6.5, 11.5 Hz, H-6), 2.26 (2H, m, H-9 and H-13), 2.50 (3H, s, CH₃-8′), 2.60 (1H, m, H-6), 3.89 (3H, s, OCH₃-5′), 4.17 (1H, dd, J=2.0, 8.0 Hz, H-3), 4.30 (2H, dd, J=12.0, 25.0 Hz, H-1), 5.97 (1H, t, J=9.0 Hz, H-5), 6.36 (1H, s, H-4′), 12.06 (1H, s, OH-3′);

¹³C NMR (CDCl₃, 125 MHz) δ21.1 (CH₃-8), 24.6 (CH₃-8′), 26.9 (CH₃-14), 29.5 (CH₃-15), 38.8 (C-7), 40.1 (C-11), 40.8 (C-10), 46.2 (C-12), 46.5 (C-6), 47.3 (C-9), 49.9 (C-13), 56.2 (OCH₃-5′), 58.9 (C-1), 70.6 (C-5), 74.5 (C-3), 106.3 (C-2′), 106.6 (C-4′), 107.3 (C-6′), 133.6 (C-2), 141.4 (C-7′), 142.4 (C-4), 158.9 (C-5′), 159.8 (C-3′), 170.6 (C-1′); and

ESIMS m/z (%): 475 (30), 473 [M+Na]⁺ (100).

CH-205-G-2 (melleolide S)
((2R,2aR,3R,4S,4aR,7aS,7bR)-decahydro-2,2a,4-trihyd roxy-6,6,7b-trimethyl-1H-cyclobuta[e]inden-3-yl)methyl
3-chloro-6-hydroxy-4-methoxy-2-methylbenzoate

¹H NMR (acetone-d₆, 500 MHz) δ0.98 (3H, s, CH₃-15), 1.07 (3H, s, CH₃-8), 1.10 (3H, s, CH₃-14), 1.45 (2H, m, H₂-10), 1.53 (3H, m, H-6, and, H₂-12), 1.72 (1H, t, J=8.5 Hz, H-6), 1.96 (1H, m, H-13), 2.08 (1H, m, H-9), 2.40 (1H, m, H-2), 2.58 (3H, s, CH₃-8′), 3.78 (1 H, t, J=11.0 Hz, H-3), 4.20 (1 H, t, J=8.5 Hz, H-5), 4.72 (2H, m, H₂-1), 6.46 (1H, s, H-4′):

¹³C NMR (acetone-d₆, 125 MHz) δ18.9(C-8′), 22.5(C-8), 32.4(C-15), 32.7(C-14), 36.6(C-11), 36.9(C-6), 37.6(C-7), 43.5(C-12), 43.7(C-2), 44.6(C-10), 47.8(C-13), 48.2(C-9), 56.6(OCH₃-5′), 66.1(C-1), 68.6(C-3), 73.4(C-5), 81.7(C-4), 99.6(C-4′), 110.3(C-2′), 115.2(C-6′), 139.8(C-7′), 159.3(C-5′), 160.8(C-3′), 169.0(C-5′)

FAB m/z (%): 469 [M+H]⁺; HRFAB m/z 469.1991 (calcd 469.1991 for C₂₄H₃₄O₇Cl).

CH-205-N (melleolide T)
(2R,2aS,4aR,7aR,7bR)-3-formyl-2,2a,4a,5,6,7,7a,7b-octahydro-2a,4a-dihydroxy-6,6,7b-trimethyl-1H-cyclobuta[e]inden-2-yl
3-chloro-4,6-dihydroxy-2-methylbenzoate

¹H NMR (CDCl₃, 500 MHz) δ0.93 (3H, s, CH₃-14), 1.12 (3H, s, CH₃-15), 1.25 (1H, d, J=13.5 Hz, H-10), 1.28 (3H, s, CH₃-8), 1.67 (1H, dd, J=7.0; 13.5 Hz, H-10), 1.92 (2H, br, s, H₂-12), 2.01(1H, dd, J=8.5, 11.5 Hz, H-6), 2.28 (2H, m, H-6 and H-9), 2.43 (3H, s, CH₃-8), 5.56 (1 H, t, J=8.5Hz,-5′), 6.49 (1H, s, H-4′), 6.70 (1H, s, H-3), 9.53 (1H, s, H-1), 11.09 (1H, br,s, OH-3′):

¹³C NMR (CDCl₃, 125 MHz) δ20.0(C-8′), 21.4(C-8), 30.8(C-14), 30.9(C-15), 31.7(C-6), 34.6(C-7), 37.6(C-11), 43.2(C-10), 50.3(C-9), 58.2(C-12), 75.2(C-13), 75.3(C-5), 77.8(C-4), 102.1(C-4′), 107.1(C-2′), 113.8(C-6′), 136.8(C-2), 139.0(C-7′), 153.1(C-3), 156.0(C-5′), 162.7(C-3′), 169.9(C-1′), 196.2(C-1)

ESIMS m/z (%): 475 (35), 473 [M+Na]⁺ (100).

CH-205-R (melleolide R)
(2R,2aR,3R,4S,4aR,7aS,7bR)-decahydro-2a,4-dihydroxy-3-(hydroxymethyl)-6,6,7b-trimethyl-1H-cyclobuta[e]inden-2-yl
3-chloro-4,6-dihydroxy-2-methylbenzoate

¹H NMR (acetone-d₆, 500 MHz) δ0.98 (3H, s, CH₃-15), 1.09 (3H, s, CH₃-14), 1.16 (3H, s, CH₃-8), 1.44 (2H, m, H₂-10), 1.51 (1H,dd, J=7.6,14.0 Hz, H-12), 1.85(1H, m, H-6), 1.96 (2H, m, H-6 and H-12), 2.07 (1H, m, H-2), 2.10-2.20 (2H, m, H-9 and H-13), 2.61 (3H, s, CH₃-8′), 3.73 (1 H, t, J=11.2 Hz, H-3), 3.90 (1H, dd, J=5.2, 10.8 Hz, H-1), 4.03 (1H, dd, J=4.0, 11.2 Hz, H-1), 5.33 (1H, t, J=8.4 Hz s, H-5), 6.44 (1H, s, H-4′)

¹³C NMR (acetone-d₆, 125 MHz) δ19.9(C-8′), 22.3(C-8), 32.4(C-15), 32.7(C-14), 34.4(C-6), 36.7(C-7), 39.0(C-11), 43.4(C-12), 44.4(C-10), 46.3(C-13), 47.4(C-2), 48.0(C-9), 62.8(C-1′), 68.9(C-3), 77.1(C-5), 81.3(C-4), 102.4(C-4′), 108.3(C-2′), 114.7(C-6′), 140.2(C-7′), 158.4(C-5′), 162.2(C-3′), 171.0(C-1′)

ESIMS m/z (%): 479 (30), 477 [M+Na]⁺ (100).

CH-205-K (melleolide P)
(2R,2aS,7bR)-2,2a,4a,5,6,7,7a,7b-octahydro-2a-hydroxy-3-(hydroxymethyl)-6,6,7b-trimethyl-1H-cyclobuta[e]inden-2-yl
3-chloro-4,6-dihydroxy-2-methylbenzoate

Colorless solid, mp. 68-69 ° C. ; [a]₂₅ 28° (c 1.0, MeOH);

UV (MeOH) λmax (log ε) 311 (3.72), 263 (3.91), 212 (4.42) nm;

IR (KBr) νₘₐₓ 3530, 1655, 1611, 1445, 1382, 1318, 1239, 1124 cm⁻¹;

¹H NMR (acetone-d₆, 500 MHz) δ1.00 (3H, s, CH₃-14 or CH₃-15), 1.01 (3H, s, CH₃-15 or CH₃-14), 1.29 (3H, s, CH₃-8), 1.40-1.44 (2H, m, H₂-10), 1.50 (1H, m, H-12), 1.68 (1H, m, H-6), 1.86 (1H, m, H-12), 1.99 (1H, m, H-6), 2.20 (1H, m, H-9), 2.51 (3H, s, CH₃-8′), 2.78 (1H, t, J=7.5 Hz, H-13), 4.08 (1H, d, J=12.5 Hz, H-1), 4.36 (1H, d, J=12.5 Hz, H-1), 5.55 (1H, t, J=9.0 Hz, H-5), 5.78 (1H, br s, H-3), 6.45 (1H, s, H-4′), 10.75 (1H, s, OH-3′);

¹³C NMR (acetone-d₆, 125 MHz) δ19.6 (CH₃-8′), 22.1 (CH₃-8), 32.2 (CH₃-14 or CH₃-15), 32.3 (CH₃-15 or CH₃-14), 33.8 (C-6), 39.6 (C-7), 38.4 (C-11), 39.9 (C-13), 42.0 (C-10), 45.4 (C-9), 48.4 (C-12), 65.3 (C-1), 77.3 (C-4), 80.2 (C-5), 102.5 (C-4′), 108.2 (C-2′), 114.9 (C-6′), 132.9 (C-3), 135.2 (C-2), 140.3 (C-7′), 158.5 (C-5′), 162.3 (C-3′), 170.7 (C-1′); and

ESIMS m/z (%): 459 [M+Na]⁺ (100).

CH-205-A (armillaricin)
(2R,7aS,7bR)-3-formyl-6,6,7b-trimethyl-2,5,6,7,7a-hexahydro-1H-cyclobuta[e]inden-2-yl 3-chloro-4,6-dihydroxy-2-methylbenzoate

¹H NMR (CDCl₃, 400 MHz) δ0.92 (3H, s, CH₃-14), 1,12 (3H, s, CH₃-15), 1.13 (3H, s, CH₃-8), 1.37 (1H, t, J=12.0 Hz, H-10), 1.49 (1H, m, H-10), 2.00 (1H, dd, J=7.6, 11.2 Hz, H-6), 2.04 (1H, d, J=18.0 Hz, H-12), 2.35 (1H, d, J=18.0 Hz, H-12), 2.60 (3H, s, 8′-CH₃), 2.66 (1H, dd, J=6.8, 11.2 Hz, H-6), 2.87 (1H, m, H-9), 3.89 (3H, s, 5′-OCH₃), 6.16 (1H, br s, H-3), 6.34 (1H, t, J=8.0 Hz, H-5), 6.41 (1H, s, H-4′), 9.75 (1H, s, H-1), 11.37 (1H, s, 3′—OH); and

¹³C NMR (CDCl₃, 400 MHz) δ16.4 (CH₃-8), 20.1 (CH₃-8′), 27.4 (CH₃-14), 29.2 (CH₃-15), 36.3 (C-7), 37.3 (C-11), 39.3 (C10), 40.8 (C-6), 45.6 (C-12), 48.5 (C-9), 56.3 (5′-OCH₃), 72.3 (C-5), 98.5 (C-4′), 105.5 (C-2′), 110.2 (C-3), 115.9 (C6′), 129.3 (C-2), 139.5 (C-7′), 150.2 (C-13), 160.0 (C-5′), 160.7 (C-4), 163.4 (C-3′), 170.1 (C-1′), 187.5 (C-1); EIMS m/z (%): 432 (1), 430 (3), 391 (5), 232 (4), 214 (20), 201 (40), 200 (13), 199 (100), 187(5).

CH-205-E (melledonal C)
(2R,2a6,4aR,7R,7bR)-3-formyl-2,2a,4a,5,6,7,7a,7b-octahydro-2a,4a,7-trihydroxy-6,6,7b-trimethyl-1H-cyclobuta[e]inden-2-yl
3-chloro-6-hyd roxy-4-methoxy-2-methylbenzoate

¹H NMR (CDCl₃, 400 MHz) δ1.00 (3H, s, CH₃-14), 1,16 (3H, s, CH₃-15), 1.40 (3H, s, CH₃-8), 1.85 (1H, d, J=14.4 Hz, H-6), 2.06 (1H, d, J=3.2 Hz, H-12), 2.07 (1H, d, J=2.0 Hz, H-12), 2.14 (1H, d, J=14.4 Hz, H-6), 2.41 (3H, s, CH₃-8′), 2.51 (1H, d, J=3.2 Hz, H-9), 3.73 (1H, d, J=3.2 Hz, H-10), 3.86 (3H, s, OCH₃-5′), 5.69 (1H, t, J=8.8 Hz, H-5), 6.38 (1H, s, H-4′), 6.82 (1H, d, J=0.4 Hz, H-3), 9.48 (1H, s, H-1), 11.24 (1H, s, OH-3′); and

¹³C NMR (CDCl₃, 400 MHz) δ19.8 (CH₃-8′), 20.8 (CH₃-8), 23.2 (CH₃-14), 28.1 (CH₃-15), 32.0(C-12), 35.7 (C-7), 41.2 (C-11), 54.8 (C-6), 55.1 (C-9), 56.3 (OCH₃-5′), 74.2 (C-5), 74.5 (C-13), 81.4 (C-10), 98.6 (C-4′), 106.2 (C-2′), 115.4 (C-6′), 134.6 (C-2), 139.1 (C-7′), 153.0 (C-3), 159.6 (C-5′), 162.9 (C-3′), 170.1 (C-1′), 195.9 (C-1); EIMS m/z (%): 479 [M-H]⁺ (100), 346 (18), 215 (30).

CH-205-I (armillaritin)
(2R,2a6,4aR,7aR,7bR)-3-formyl-2,2a,4a,5,6,7,7a,7b-octahydro-2a,4a-dihydroxy-6,6,7b-trimethyl-1H-cyclobuta[e]inden-2-yl
2,4-dihydroxy-6-methylbenzoate

¹H NMR (CD₃OD, 400 MHz) δ0.91 (3H, s, CH₃-14 or CH₃-15), 1.10 (3H, s, CH₃-14 or CH₃-15), 1.23 (3H, s, CH₃-8), 1.27 (1H, d, J=12.8 Hz, H-10), 1.60 (1 H, dd, J=7.6, 12.8 Hz, H-10), 1.85-1.98 (3H, m, H-6 and H₂-12), 2.25 (3H, s, CH₃-8′), 2.30 (1H, dd, J=6.4, 12.8 Hz, H-9), 2.39 (1H, dd, J=8.8, 11.2 Hz, H-6), 5.60 (1H, t, J=8.8 Hz, H-5), 6.11 (1H, d, J=2.8 Hz, H-4′ or H-6′), 6.83 (1H, d, J=1.2 Hz, H-3), 9.55 (1H, s, H-1);

¹³C NMR (CD₃OD, 400 MHz) δ22.2 (C-8), 24.7 (C-8′), 31.1 (CH₃-14 or CH₃-15), 31.3 (CH₃-14 or CH₃-15), 32.2 (C-6), 35.3 (C-11), 38.9 (C-7), 44.0 (C-10), 50.7 (C-9), 58.3 (C-12), 75.3 (C-5), 76.2 (C-13), 77.9 (C-4), 101.7 (C-4′), 105.4 (C-2′), 112.4 (C-6′), 137.2 (C-2), 144.4 (C-7′), 152.9 (C-3), 163.6 (C-5′), 166.2 (C-3′), 171.9 (C-1′), 196.9 (C-1); and

ESIMS m/z (%): 439 [M+Na]⁺.

CH-205-6-4-D (armillarinin)
(2R,2aS,4aR,7bR)-3-formyl-2,2a,4a,5,6,7,7a,7b-octahydro-2a,4a-dihydroxy-6,6,7b-trimethyl-1H-cyclobuta[e]inden-2-yl
3-ch loro-6-hydroxy-4-methoxy-2-methylbenzoate

¹H NMR (acetone-d₆, 400 MHz) δ0.91 (3H, s, CH₃-14), 1.10 (3H, s, CH₃-15), 1.24 (3H, s, CH₃-8), 1.30 (1H, d, J=12.8 Hz, H-10), 1.63 (1H, dd, J=7.6, 12.8 Hz, H-10), 1.65-2.00 (3H, m, H-6 and H₂-12), 2.30-2.35 (1H, m, H-9), 2.41 (3H, s, CH₃-8′), 2.54 (1H, dd, J=8.8, 11.2 Hz, H-6), 3.89 (3H, s, OCH₃-5′), 5.58 (1H, t, J=8.8 Hz, H-5), 6.46 (1H, s, H-4′), 6.94 (1H, d, J=1.2 Hz, H-3), 9.63 (1H, s, H-1), 11.2 (1H, s, OH-3′);

¹³C NMR (acetone-d₆, 400 MHz) δ19.8 (C-8′), 22.0 (C-8), 30.6 (CH₃-14), 31.2 (CH₃-15), 31.9 (C-6), 34.7 (C-11), 38.5 (C-7), 43.6 (C-10), 50.4 (C-9), 56.7 (OCH₃-5′), 58.1 (C-12), 75.7 (C-13), 76.2 (C-5), 77.5 (C-4), 99.3 (C-4′), 107.5 (C-2′), 115.5 (C-6′), 136.7 (C-2), 139.5 (C-7′), 153.4 (C-3), 160.2 (C-5′), 163.2 (C-3′), 170.6 (C-1′), 197.0 (C-1); and

ESIMS m/z (%): 487 [M+Na]⁺.

### CH-205-L (Dihydromelleolide, Melleolide F)

(2R,2aS,7bR)-2,2a,4a,5,6,7,7a,7b-octahydro-2a-hydroxy-3-(hydroxymethyl)-6,6,7b-trimethyl-1H-cyclobuta[e]inden-2-yl
2,4-dihydroxy-6-methylbenzoate

¹H NMR (CDCl₃, 500 MHz) δ0.97 (3H, s,'CH₃-15), 0.98 (3H, s, CH₃-14), 1.24 (3H, s, CH₃-8), 1.31 (1H, d, J=12.5 Hz, H-10), 1.38 (1H, dd, J=6.5, 12.5 Hz, H-10), 1.46 (1H, d, J=13.0 Hz, H-12), 1.63 (1H, t, J=10.5 Hz, H-6), 1.82 (1H, dd, J=8.5, 13.5 Hz, H-12), 1.92 (1H, dd, J=9.0, 11.0 Hz, H-6), 2.14 (1H, m, H-9), 2.27 (3H, s, CH₃-8′), 2.72 (1H, br t, J=7.5 Hz, H-13), 3.99 (1H, d, J=12.0 Hz, H-1), 4.27 (1H, d, J=12.0 Hz, H-1), 5.52 (1H, t, J=9.0 Hz, H-5), 5.76 (1H, br s, H-3), 6.13 (1H, d, J=2.0 Hz, H-6′), 6.21 (1H, d, J=2.0 Hz, H-4′);

¹³C NMR (CDCl₃, 125 MHz) δ21.1 (CH₃-8), 24.0 (CH₃-8′), 31.7 (CH₃-14), 31.9 (CH₃-15), 32.4 (C-6), 38.0 (C-11), 38.7 (C-13), 39.0 (C-7), 41.3 (C-10), 44.1 (C-9), 47.4 (C-12), 66.0 (C-1), 77.6 (C-4), 78.3 (C-5), 101.3 (C-4′), 104.6 (C-2′), 111.9 (C-6′), 132.4 (C-2), 136.2 (C-3), 143.8 (C-7′), 161.4 (C-5′), 165.1 (C-3′), 171.3 (C-1′); and

ESIMS m/z (%): 425 [M+Na]⁺ (100).

### CH-205-O (Armillaridin)

(2R,2aS,7bR)-3-formyl-2,2a,4a,5,6,7,7a,7b-octahydro-2a-hydroxy-6,6,7b-trimethyl-1 H-cyclobuta[e]inden-2-yl
3-chloro-6-hydroxy-4-methoxy-2-methylbenzoate

¹H NMR (CDCl₃, 400 MHz) δ0.98 (3H, s, CH3-15), 1.01 (31-1, s, CH3-14), 1.25 (1H, d, J=12.8 Hz, H-10), 1.31 (3H, s, CH3-8), 1.48 (1H, dd, J=6.8, 12.8 Hz, H-10), 1.53-1.58 (2H, m, H-6 and H-12), 2.00 (1H, dd, J=9.6, 13.6 Hz, H-6), 2.06 (1H, dd, J=8.8, 11.2 Hz, H-12), 2.26 (1H, m, H-9), 2.42 (3H, s, CH3-8′), 3.00 (1H, br t, J=2.0 Hz, H-13), 3.86 (3H, s, OCH3-5′), 5.61 (1H, t, J=8.8 Hz,

H-5), 6.38 (1H, s, H-4′), 6.78 (1H, d, J=2.0 Hz, H-3), 9.45 (1H, s, H-1), 11.33 (1H, s, OH-3′);

13C NMR (CDCl3, 100 MHz) δ19.8 (CH3-8′), 21.1 (CH3-8), 31.1 (CH3-14), 31.5 (CH3-15), 33.1(C-12), 37.6 (C-11), 38.0 (C-7), 40.3 (C-13), 41.7 (C-10), 44.1 (C-9), 46.6 (C-6), 56.3 (OCH3-5′), 75.0 (C-4), 77.7 (C-5), 98.6 (C-4′), 106.3 (C-2′), 115.4 (C-6′), 137.3 (C-2), 139.1 (C-7′), 158.3 (C-3), 159.5 (C-5′), 162.9 (C-3′), 170.2 (C-1′), 195.9 (C-1); and

ESIMS m/z (%): 471 [M+Na]⁺ (100), 437 (75).

### CH-205-O-1 (Armillarin)

(2R,2aS,7bR)-3-formyl-2,2a,4a,5,6,7,7a,7b-octahydro-2a-hydroxy-6,6,7b-trimethyl-1H-cyclobuta[e]inden-2-yl2-hydroxy-4-methoxy-6-methylbenzoate

¹H NMR (CDCl₃, 500 MHz) δ0.98 (3H, s, CH3-15), 1.01 (3H, s, CH3-14), 1.26 (1H, d, J=10.0 Hz, H-10), 1.31 (3H, s, CH3-8), 1.48 (1H, m, H-10), 1.55 (2H, m, H-6 and H-12), 1.97-2.06 (2H, m, H-6 and H-12), 2.26 (1H, m, H-9), 2.28 (3H, s, CH3-8′), 3.00 (1H, br t, J=6.0 Hz, H-13), 3.78 (3H, s, OCH3-5′), 5.63 (1H, t, J=7.2 Hz, H-5), 6.18 (1H, br s, H-4′), 6.29 (1H, br s, H-6′), 6.77 (1H, br s, H-3), 9.45 (1H, d, J=1.2 Hz, H-1), 11.64 (1H, s, OH-3′);

13C NMR (CDCl3, 125 MHz) δ21.2 (CH3-8′), 24.5 (CH3-8), 31.1 (CH₃-14), 31.6 (CH₃-15), 33.1(C-12), 37.5 (C-11), 38.0 (C-7), 40.3 (C-13), 41.7 (C-10), 44.1 (C-9), 46.6 (C-6), 55.3 (OCH₃-5′), 75.1 (C-4), 77.2 (C-5), 98.8 (C-4′), 105.0 (C-2′), 111.1 (C-6′), 137.5 (C-2), 142.5 (C-7′), 158.1 (C-3), 163.9 (C-5′), 165.7 (C-3′), 170.8 (C-1′), 195.8 (C-1); and

ESIMS m/z (%): 437 [M+Na]⁺ (100).

### CH-205-P (Armillarikin)

(2R,2aS,7R,7bR)-3-formyl-2,2a,4a,5,6,7,7a,7b-octahydro-2a,7-dihydroxy-6,6,7b-trimethyl-1H-cyclobuta[e]inden-2-yl
3-chloro-6-hydroxy-4-methoxy-2-methylbenzoate

¹H NMR (acetone-d₆, 400 MHz) δ0.99 (3H, s, CH₃-15), 1.02 (3H, s, CH₃-14), 1.31 (3H, s, CH₃-8), 1.57 (1H, dd, J=6.0, 12.8 Hz, H-12), 1.71 (1H, d, J=8.8, 10.8 Hz, H-6), 2.05-2.11 (2H, m, H-6 and H-12), 2.42 (3H, s, CH₃-8′), 2.47 (1 H, dd, J=3.2, 10.0 Hz, H-9), 3.20 (1 H, m, H-13), 3.62 (1 H, d, J=3.2 Hz, H-10), 3.91 (3H, s, OCH₃-5′), 5.75 (1H, t, J=8.8 Hz, H-5), 6.50 (1H, s, H-4′), 7.03 (1H, d, J=2.8 Hz, H-3), 9.48 (1H, s, H-1), 11.16 (1H, s, OH-3′);

¹³C NMR (acetone-d₆, 400 MHz) δ19.7 (CH₃-8′), 21.3 (CH₃-8), 24.0 (CH₃-14), 28.4 (CH₃-15), 33.4(C-6), 36.5 (C-7), 36.7 (C-13), 43.3 (C-11), 43.9 (C-12), 47.7 (C-9), 56.8 (OCH₃-5′), 75.0 (C-4), 76.7 (C-5), 81.4 (C-10), 99.4 (C-4′), 108.2 (C-2′), 112.0 (C-6′), 136.3 (C-2), 139.6 (C-7′), 156.8 (C-3), 160.3 (C-5′), 163.2 (C-3′), 170.5 (C-1′), 195.4 (C-1); and

ESIMS m/z (%): 487 [M+Na]⁺ (100).

CH-205-Q (melledonal B)
(2R,2aS,4aR,7R,7bR)-3-formyl-2,2a,4a,5,6,7,7a,7b-octahydro-2a,4a,7-trihydroxy-6,6,7b-trimethyl-1H-cyclobuta[e]inden-2-yl
3-chloro-4,6-dihydroxy-2-methyl benzoate

¹H NMR (acetone-d₆, 400 MHz) δ0.98 (3H, s, CH₃-15), 1.16 (3H, s, CH₃-14), 1.40 (3H, s, CH₃-8), 1.91-2.02 (3H, m H-6 and H₂-12), 2.26 (1H, dd, J=8.8, 10.4 Hz, H-6), 2.42 (3H, s, CH₃-8′), 2.54 (1H, d, J=4.0 Hz, H-9), 3.74 (1H, br s, H-10), 5.70 (1H, t, J=8.8 Hz, H-5), 6.44 (1H, s, H-4′), 6.95 (1H, d, J=0.8 Hz, H-3), 9.59 (1H, s, H-1), 10.91 (1H, br s, OH-3′);

¹³C NMR (acetone-d₆, 400 MHz) δ19.7 (CH₃-8′), 21.4 (CH₃-8), 24.0 (CH₃-14), 28.4 (CH₃-15), 32.8(C-6), 36.9 (C-7), 41.8 (C-11), 55.1 (C-12), 55.4 (C-9), 75.0 (C-5), 75.1 (C-13), 76.8 (C-4), 82.1 (C-10), 102.5 (C-4′), 108.2 (C-2′), 114.7 (C-6′), 134.8 (C-2), 140.0 (C-7′), 150.9 (C-3), 158.5 (C-5′), 162.3 (C-3′), 170.3 (C-1′), 195.6 (C-1); and

ESIMS m/z (%): 491 (30), 489 [M+Na]⁺ (100).

### CH-205-S-1 (Melleolide B)

(2R,2aS,7R,7bR)-2,2a,4a,5,6,7,7a,7b-octahydro-2a,7-dihydroxy-3-(hydroxymethyl)-6,6,7b-trimethyl-1H-cyclobuta[e]inden-2-yl
2-hydroxy-4-methoxy-6-methyl benzoate

¹H NMR (CDCl₃, 500 MHz) δ0.97 (3H, s, CH₃-15), 1.02 (3H, s, CH₃-14), 1.35 (3H, s, CH₃-8), 1.46 (1H, dd, J=5.5, 13.0 Hz, H-12), 1.64 (1H, t, J=10.0 Hz, H-6), 1.93-1.97 (2H, m, H-6 and H-12), 2.25 (1H, dd, J=3.5, 9.5 Hz. H-9), 2.34 (3H, s, CH₃-8′), 2.81 (1H, br s, H-13), 3.58 (1H, d, J=3.5 Hz, H-10), 3.77 (3H, s, OCH₃-5′), 3.91 (1H, d, J=12.5 Hz, H-1), 4.21 (1H, d, J=12.5 Hz, H-1), 5.66 (1 H, t, J=9.0 Hz, H-5), 5.83 (1 H, br s, H-3), 6.22 (1 H, d, J=1.5 Hz, H-6′), 6.27 (1 H, d, J=1.5 Hz, H-4′), 11.60 (1 H, s, OH-3′);

¹³C NMR (CDCl₃, 125 MHz) δ21.2 (CH₃-8), 23.8 (CH₃-15), 24.1 (CH₃-8′), 29.1 (CH₃-14), 32.7(C-6), 34.9 (C-13), 36.3 (C-7), 42.5 (C-11), 44.5 (C-12), 46.9 (C-9), 55.3 (OCH₃-5′), 76.4 (C-4), 76.7 (C-5), 82.4 (C-10), 98.8 (C-4′), 104.9 (C-2′), 111.2 (C-6′), 132.7 (C-2), 135.8 (C-3), 142.9 (C-7′), 164.1 (C-5′), 165.7 (C-3′), 171.0 (C-1′); and

ESIMS m/z (%): 455 [M+Na]⁺ (100).

### CH-205-S-2 (Melleolide I)

(2R,2a5,7R,7bR)-2,2a,4a,5,6,7,7a,7b-octahydro-2a,7-dihydroxy-3-(hydroxymethyl)-6,6,7b-trimethyl-1H-cyclobuta[e]inden-2-yl-3-chloro-6-hydroxy-4-methoxy-2-methylbenzoate

¹H NMR (acetone-d₆, 400 MHz) δ0.96 (3H, s, CH₃-15), 1.01 (3H, s, CH₃-14), 1.36 (3H, s, CH₃-8), 1.46 (1H, dd, J=6.5, 13.2 Hz, H-12), 1.71 (1H, dd, J=8.8, 10.0 Hz, H-6), 1.92 (1H, dd, J=10.0, 12.8 Hz, H-6), 1.99 (1H, d, J=8.8 Hz, H-6), 2.34 (1H, dd, J=4.0, 9.6 Hz, H-9), 2.49 (3H, s, CH₃-8′), 2.86 (1H, m, H-13), 3.57 (1H, d, J=4.0 Hz, H-10), 3.91 (3H, s, OCH₃-5′), 4.03 (1H, d, J=13.2 Hz, H-1), 4.27 (1H, d, J=13.2 Hz, H-1), 5.61 (1H, t, J=8.8 Hz, H-5), 5.86 (1H, d, J=0.8 Hz, H-3), 6.51 (1H, s, H-4′);

¹³C NMR (acetone-d₆, 100 MHz) δ19.4 (CH₃-8′), 21.8 (CH₃-8), 24.4 (CH3-15), 29.2 (CH₃-14), 33.8 (C-6), 35.7 (C-13), 37.4 (C-7), 43.3 (C-11), 45.7 (C-12), 47.7 (C-9), 56.8 (OCH₃-5′), 64.3 (C-1), 76.5 (C-4), 78.1 (C-5), 82.2 (C-10), 99.4 (C-4′), 108.5 (C-2′), 115.6 (C-6′), 133.0 (C-3), 133.8 (C-2), 139.6 (C-7′), 160.1 (C-5′), 162.5 (C-3′), 170.5 (C-1′); and

ESIMS m/z (%): 489 [M+Na]⁺ (100), 491 (40).

### Example 2

### Cytotoxity of Compounds of Example 1.2 on Cancer Cells

The alamar blue (AB) assay (dye purchased from Biosource International, Nivelles, Belgium) was used to estimate cell viability in accordance with procedures previously described by Fatokun et al (Bone (2006) 39, 542-551). Briefly, cells were treated with various concentration of each of the test compounds (e.g., compounds of example 1.1) at 37° C. for 48 hours, and the medium in each well was then aspirated at the end of the treatment, 100 μL of fresh medium containing 10% v/v AB was then added to the control and the treated wells, respectively. Plates were then incubated at 37° C. for 6 hour prior to measuring the absorbance at 570 nm and at 600 nm wavelengths using a spectrophotometric plate reader (DYNEX Technologies, USA). Experimental data were normalized to control values. Results are summarized in Table 1. CH-205-A, K, L, O, and -P were effective on MCF-7 cells; CH-205-A, H, K, L, O, and -P were effective on H460 cells; CH-205-A and -P were effective on HT-29 cells and CH-205-A, H, N, and -O were effective on CEM cells.

TABLE 1
Cytotoxicity of compounds of Example 1.1

IC₅₀ (μM)

| Compounds | MCF-7 | H460 | HT-29 | CEM |
|---|---|---|---|---|
| CH-205-A | 4.8 | 5.5 | 4.6 | 5.8 |
| CH-205-E | >100 | >100 | 85.6 | 49.6 |
| CH-205-H | 56.5 | 5.5 | 7.1 | 5.4 |
| CH-205-K | 4.8 | 4.5 | 56.7 | 28.8 |
| CH-205-L | 8.3 | 5.1 | 58.4 | 41.2 |
| CH-205-N | >100 | >100 | 32.1 | 5.5 |
| CH-205-O | 1.7 | 4.5 | 42.1 | 5.1 |
| CH-205-P | 4.4 | 5.7 | 34.7 | 44.6 |
| CH-205-Q | >100 | >100 | >100 | >100 |

### Example 3

### Anti-Angiogenic Effects of Compounds of Example 1.2

### 3.1 In Vitro Angiogenesis Assay

Anti-angiogenic effects of CH-205 compounds were estimated by their inhibition on the growth of endothelial cells. The toxicity of CH-205 compounds to endothelial cells was estimated using MTT (3-(4,5-dimethy;thiazol-2-yl)-2,5-diphenyltetrazolium bromide, a tetrazole) assay. MTT assay is a standard colorimetric assay (an assay which measures changes in color) for measuring the activity of enzymes that reduce yellow MTT to formazan, giving a purple color. It is also used to determine cytotoxity of potential medicinal agents and other toxic materials, since those agents would result in cell toxicity and therefore metabolic dysfunction and therefore decreased performance in the assay. The cytotoxity concentrations of the CH-205 compounds measured in MTT test are all over 20 μM (Table 2), however, their effects on angiogenesis inhibition are in nM range, indicating the safety of these compounds (Table 3).

TABLE 2
Cell viability of CH-205 compounds on ECs (% of control)

| | Cell viability (% of control) | | | | | | |
|---|---|---|---|---|---|---|---|
| | 1 | 10 | 50 | 100 | 250 | 500 | |
| Compounds | μM | μM | μM | μM | μM | μM | IC₅₀ |
| CH-205-A | 101.4 | 87.9 | 17.7 | 8.4 | 7.1 | 7.9 | 24.1 |
| CH-205-E | 93.5 | 98.6 | 80.8 | 63.7 | 32.1 | 7.4 | 220.5 |
| CH-205-H | 99.7 | 128.7 | 34.9 | 12.7 | 8.4 | 7.0 | 29.3 |
| CH-205-K | 103.7 | 122.7 | 6.4 | 6.4 | 7.0 | 8.8 | 21.4 |
| CH-205-L | 93.2 | 118.8 | 6.5 | 6.4 | 6.5 | 9.8 | 21.1 |
| CH-205-N | 96.5 | 94.1 | 96.5 | 85.1 | 55.2 | 7.6 | 270.5 |
| CH-205-O | 98.5 | 43.6 | 7.0 | 6.5 | 7.1 | 7.0 | 13.2 |
| CH-205-P | 105.1 | 78.5 | 6.8 | 9.8 | 6.8 | 9.6 | 20.7 |
| CH-205-Q | 104.7 | 98.9 | 105.5 | 108.6 | 100.2 | 74.1 | >500 |

TABLE 3
In vitro antiangiogenesis of CH-205 compounds on ECs

| Compounds | IC₅₀ (nM) |
|---|---|
| CH-205-A | 40.3 |
| CH-205-E | 23.0 |
| CH-205-H | 1.3 |
| CH-205-K | 3.2 |
| CH-205-L | 1.1 |
| CH-205-N | 3.5 |
| CH-205-O | 2.1 |
| CH-205-P | 1.8 |
| CH-205-Q | 1.0 |

### 3.2 Cell Migration and In Vivo Angiogenesis Assay

Cell migration is involved in angiogenesis. Therefore, CH-205 compounds were tested for their inhibition on edothelial cell migration ability using wound healing assay.

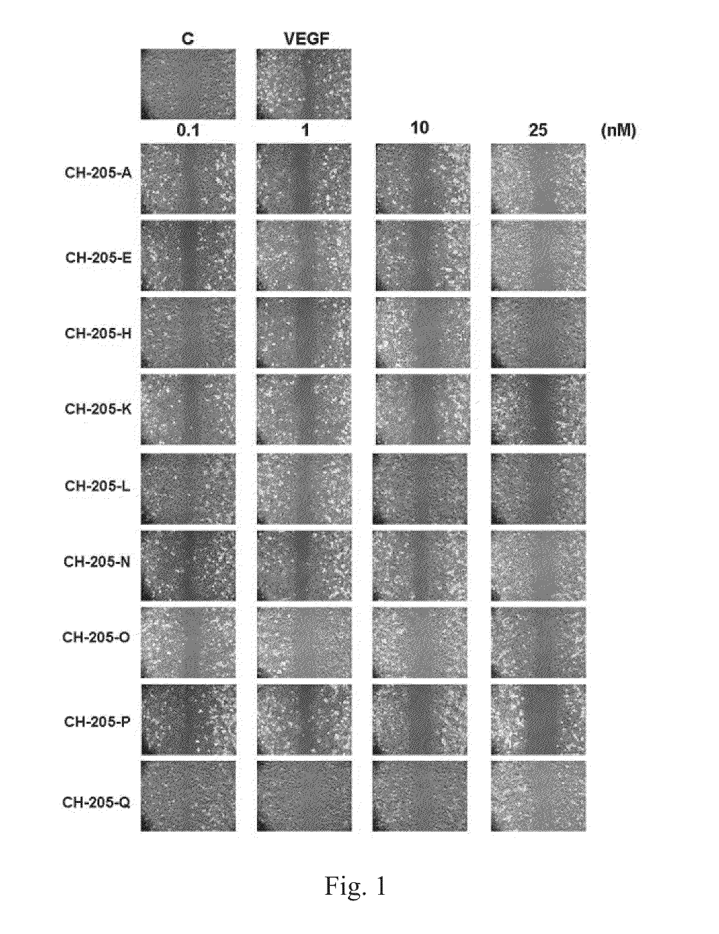

Wound Healing Assay Vascular endothelial cell migration was determined by means of a wound healing migration assay using a commercial product, Culture-Insert (iBidi GmbG, Germany). A scratch of 0.5 mm in width was made according to the manufacturer's manual, that is, by scraping ECs from the middle of the coverslip, leaving a 500 μm area devoid of cells. ECs were washed and cultured for 4 h with or without vascular endothelial growth factor (VEGF) and pretreatment with serial dilution of various CH-205 compounds, including CH-205-A, CH-205-E, CH-205-H, CH-205-K, CH-205-K, CH-205-L, CH-205-N, CH-205-O, CH-205-P and CH-205-Q. The migration of endothelial cells was visualized with an inverted Nikon/TMS microscope at a magnification of ×10. The cells that had migrated across the edge of the wound were photo is recorded under a microscope. Results are illustrated in FIG. 1. After application of VEGF for 4 h, the width was narrowed due to the migration of ECs. CH-205 compounds pretreatment dose-dependently inhibited VEGF-induced endothelial cell migration.

### Example 4

### In Vivo Anti-Tumor Effects of Compounds of Example 1.2

### 4.1 In Vivo Tumor Cells Xenograft

The established, transplantable, 5×10⁶/200 μL H460 tumor cells were prepared in RPMI medium. The 200 μL of cell suspension was injected subcutaneously into right flank area of each mouse. The mice were randomized into vehicle control and drug treatment groups of four-six animals each when xenografts were palpable with a tumor size of 40 mm³. Adminitration of the lead compounds or vehicle began on day 15 when the median tumor size was ranged from 75 to 80 mm³. Tumor-bearing animals were randomized (five per group) prior to treatment. The candidate test drugs were intraperitoneal (IP) administered into with optimal dosage once every other day (48 hr apart) for 7 or 10 times. Test drug CH-205-O (4, 40, 80 mg/kg) was given 10 times and CH-205-K (4, 20, 40, 80 mg/kg) was given 7 times. Solvent vehicle and cisplatin (5 mg/kg) were used to administrate, respectively, as io negative and positive controls. Two days after last administration of tested compounds, the animals were sacrificed. The tumor dimensions were measured every other day using a linear caliper and tumor volume is calculated using the equation V (mm³)=a×b²/2, where a is the largest diameter and b is the smallest diameter. Body weights are also recorded once weekly. Tumor is growth inhibition ratio (TGI) is calculated using the equation TGI=(1−(the mean treated tumor mass/mean control tumor mass))*100%.

### 4.2 Anti-Tumor Effects of CH-250-O Against H460 Xenograft

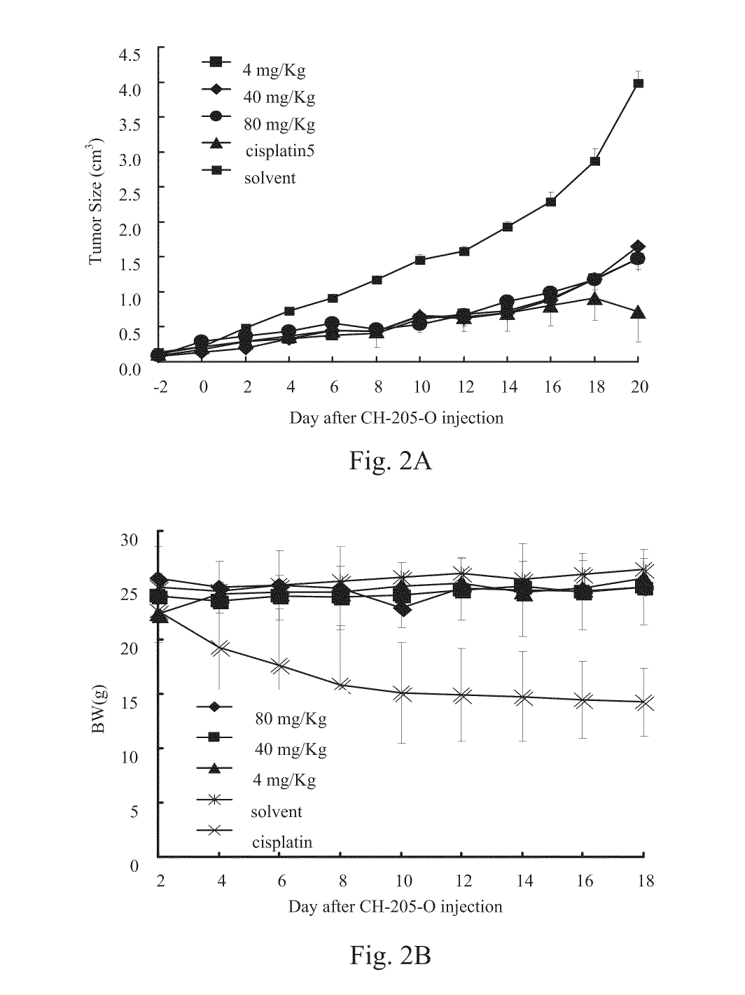

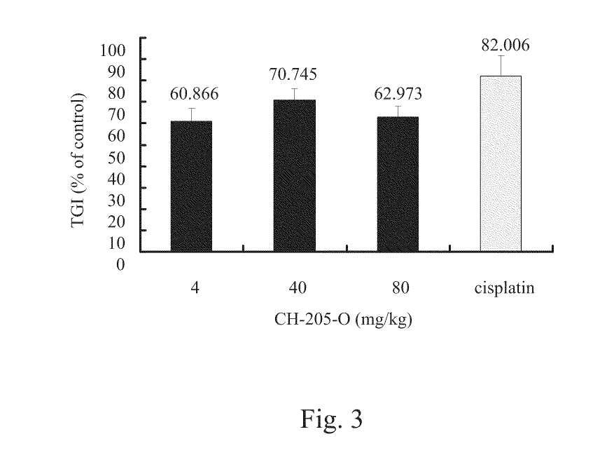

FIGS. 2A, 2B and 3 illustrate the effects of CH-205-P on H460 lung cancer tumor xenograft. In this study, mice were injected s.c. with 5×10⁶ H460 cells (day 0), and treatments started on day 16, data presented are mean±SEM (n=4-6/group). Results indicated that the group of mice that received CH-205-O at 4, 40 and 80 mg/kg each other day resulted in a highly significant tumor regression occurring after 10 times of CH-205-O injection. After sacrificed, the tumor volumes were found to be 1.475±0.155, 1.470±0.177, 1.645±0.206, and 3.984±0.484 cm³ in 4, 40, 80 mg/kg of CH-205-O and solvent control treated mice, respectively (FIG. 2A). There were no different effect on body weight between CH-205-O treated groups and control groups. The mice received cisplatin injected have decreased effect on body weight growth (FIG. 2-B). The TGI of CH-205-O at 4, 40, 80 mg/kg and cisplatin treated mice was 60.87%, 70.7%, 62.9%, and 82%, respectively (FIG. 3).

### 4.3 Anti-Tumor Effects of CH-205-K Against H460 Xenograft

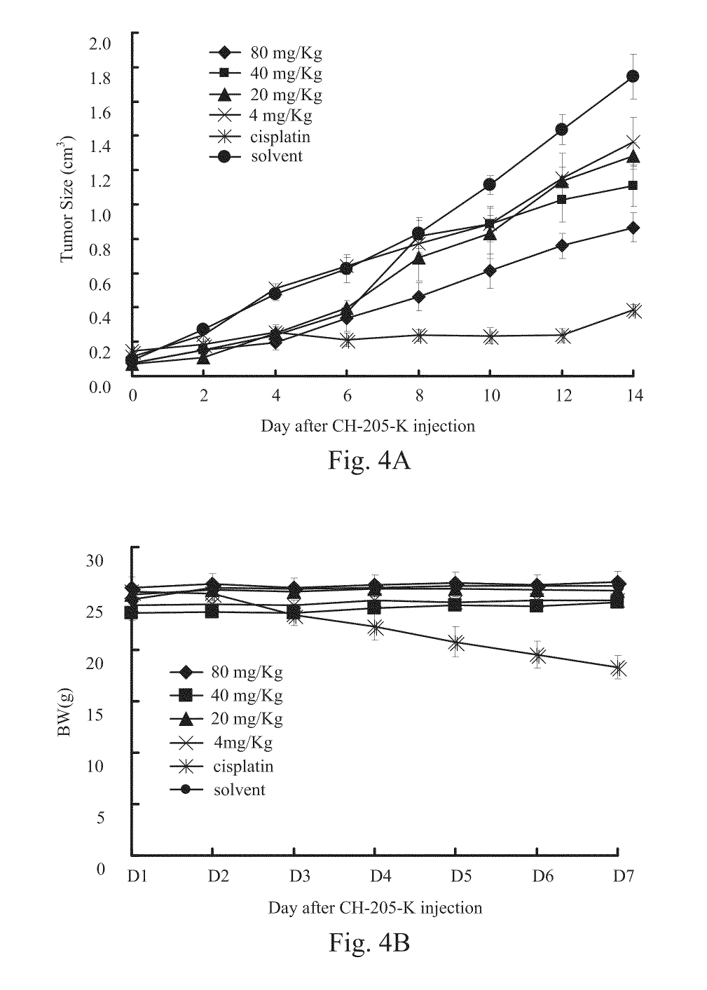

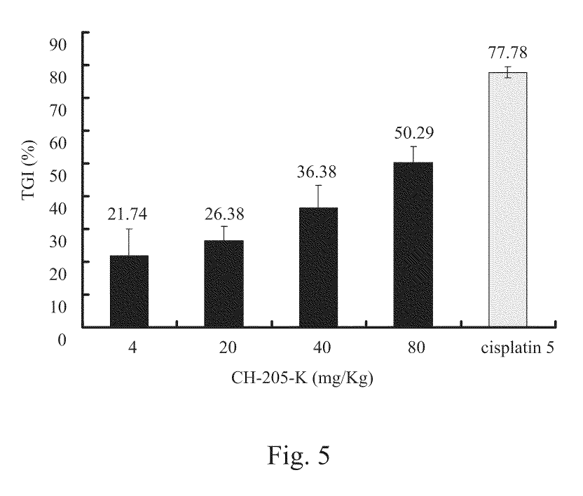

FIGS. 4A, 4B and 5 illustrate the effects of CH-205-K on H460 lung cancer tumor xenograft. Mice were injected with 5×10⁶ H460 cells in accordance with procedure described above. Results indicated that CH-205-K injection showed similar dose-dependent tumor regression at 4, 20, 40 and 80 mg/kg. After sacrificed, the tumor volumes were found to be 1.364±0.14, 1.283±0.077, 1.109±0.119, 0.867±0.086 and 1.743±0.129 cm³ in 4, 20, 40, 80 mg/kg of CH-205-K and control treated mice, respectively (FIG. 4A). There are no different effect on body weight between CH-205-K treated groups and control groups. The mice received cisplatin injected have significantly decreased effect on body weight growth (FIG. 4-B). The TGI of CH-205-K treated mice at 4, 20, 40, 80 mg/kg were 21.74%, 26.38%, 36.38%, 50.29%, and 77.78%, respectively (FIG. 5).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A protoilludane norsesquiterpenoid ester compound having a formula of:

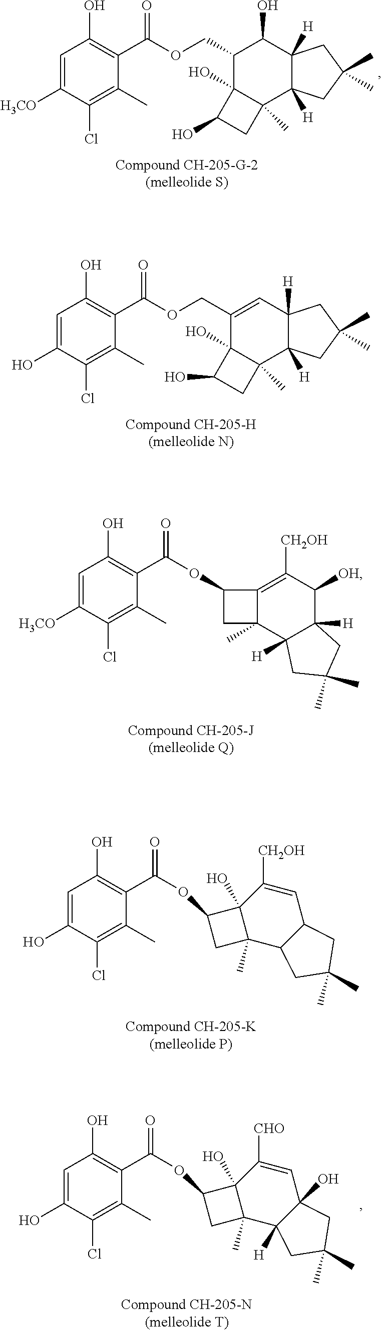

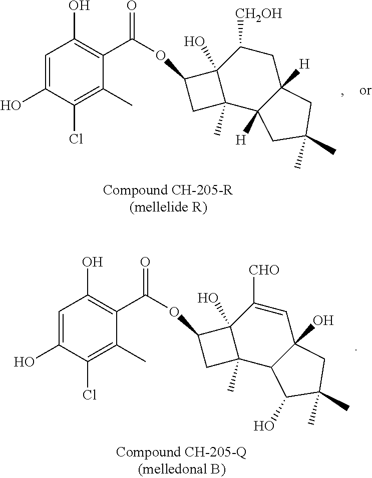

2. A pharmaceutical composition for treating a proliferative disease, comprising a pharmaceutically acceptable excipient and a therapeutically effective amount of the protoilludane norsesquiterpenoid ester compound of claim 1.

3. The composition of claim 2, further comprising a chemotherapeutic agent selected from the group consisting of 5-fluorouracil, vinblastin, doxorubicin and cisplatin.

4. The composition of claim 2, wherein the proliferative disease is any of a breast cancer, a lung cancer, a colon cancer or leukemia.

5. A pharmaceutical composition for treating a proliferative disease, comprising a pharmaceutically acceptable excipient and a therapeutically effective amount of a protoilludane norsesquiterpenoid ester compound having a formula of,

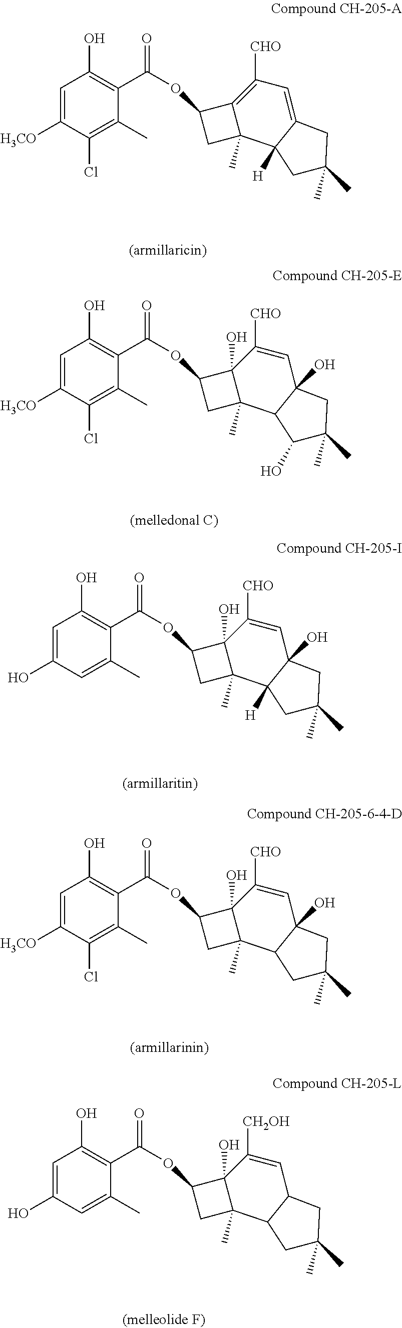

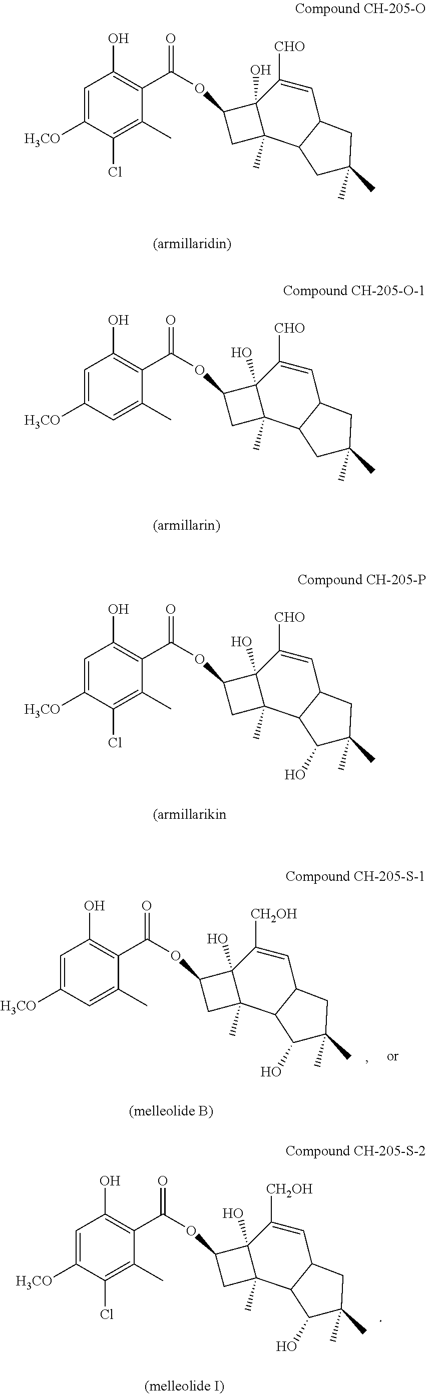

6. The composition of claim 5, wherein the proliferative disease is any of a breast cancer, a lung cancer, a colon cancer or leukemia.

7. The composition of claim 5, further comprising a chemotherapeutic agent selected from the group consisting of 5-fluorouracil, vinblastin, doxorubicin and cisplatin.

8. A method of isolating a protoilludane norsesquiterpenoid ester compound comprising:
extracting the mycelia of Armillaria mellea with an organic solvent;
partitioning the organic solvent extract with a mixture of ethyl acetate and water to produce an ethyl acetate layer and an aqueous layer;
separating the protoilludane norsesquiterpenoid ester compound from the ethyl acetate layer by high-performance liquid chromatography (HPLC) with a mixture of n-hexane and ethyl acetate in a ratio from about 20:1 to about 0:1.

9. The method of claim 8, wherein the organic solvent is ethanol.

10. The method of claim 8, wherein the ratio of the mixture of n-hexane and ethyl acetate is about 5:1.

11. The method of claim 8, wherein the ratio of the mixture of n-hexane and ethyl acetate is about 3:1.

12. The method of claim 8, wherein the ratio of the mixture of n-hexane and ethyl acetate is about 1:1

13. The method of claim 8, wherein the protoilludane norsesquiterpenoid ester compound is selected from the group consisting of: 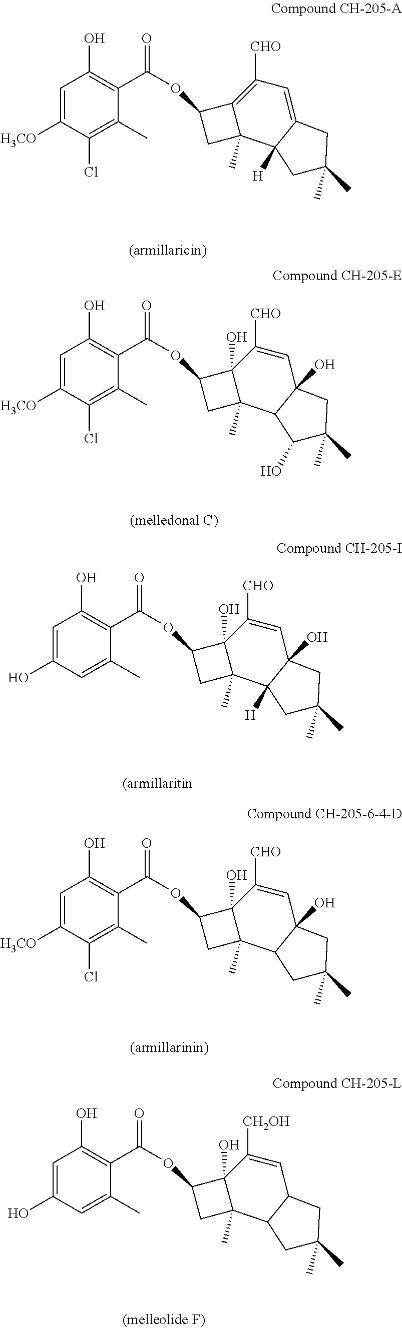 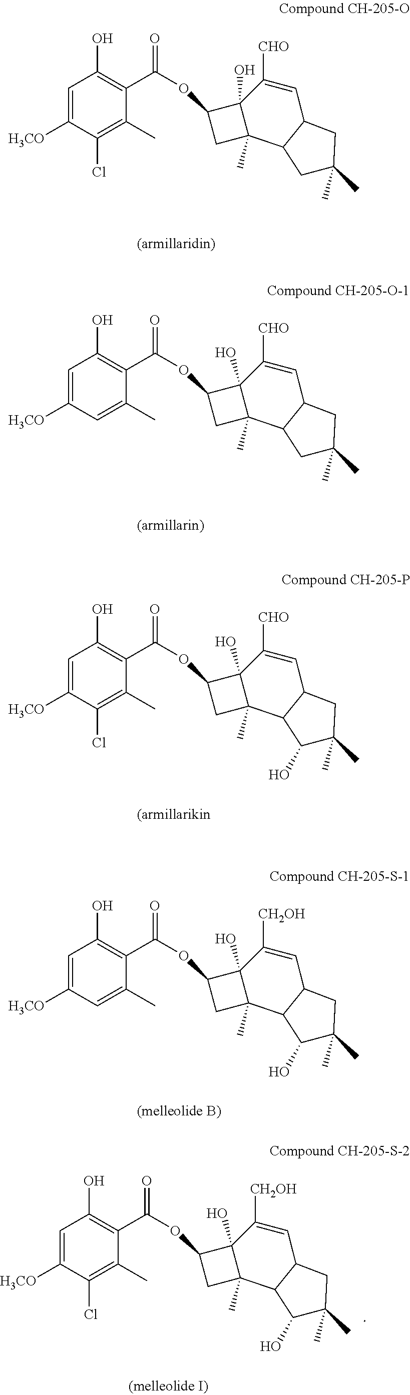 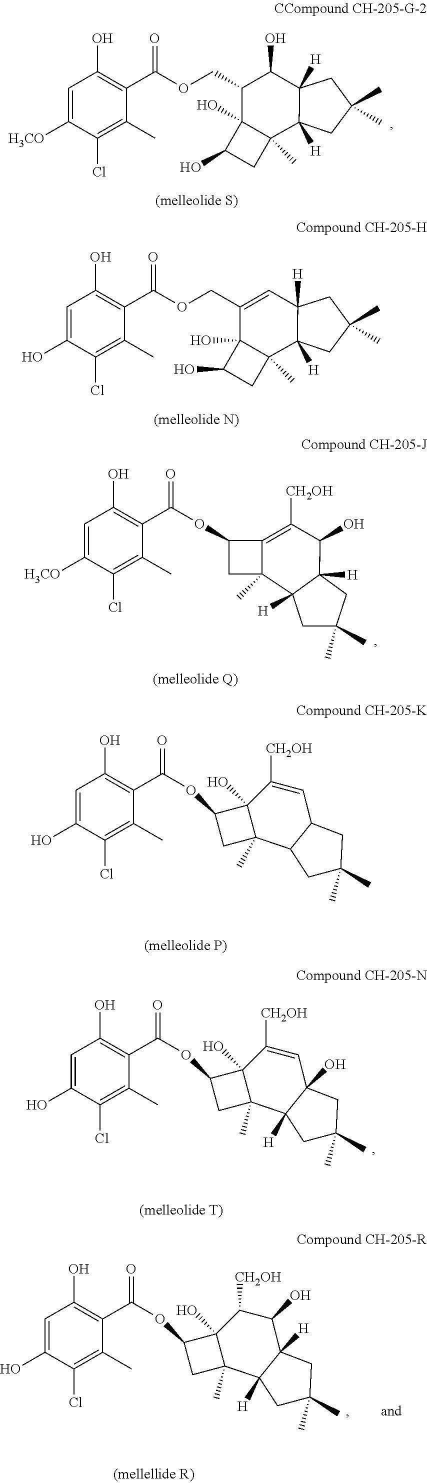 [chemical structure]
